(19) United States
(12) Patent Application Publication
(10) Pub. No.: US 2006/0020277 A1
(43) Pub. Date: Jan. 26, 2006

(54) Gastric reshaping devices and methods

(76) Inventors: Christopher J. Gostout, Rochester, MN (US); Elizabeth Rajan, Rochester, MN (US); Amy E. Foxx-Orenstein, Rochester, MN (US); Joseph A. Murray, Rochester, MN (US); Michael Camilleri, Rochester, MN (US)

Correspondence Address: MERCHANT & GOULD PC, P.O. BOX 2903, MINNEAPOLIS, MN, 55402-0903, US

(21) Appl. No.: 11/185,213

(22) Filed: Jul. 19, 2005

Related U.S. Application Data
(60) Provisional application No. 60/589,481, filed on Jul. 20, 2004.

(51) Int. Cl. A61B 17/08

(52) U.S. Cl. 606/153

(57) ABSTRACT

Gastric volume of a patient is reduced by deploying an endoscope into a stomach through the esophagus. A plurality of anterior anchors are affixed to the anterior wall of the stomach. The anterior anchors are distributed along an anterior line of the stomach wall beginning near the cardia region and extending toward the stomach exit. A plurality of posterior anchors are affixed to the posterior wall of the stomach. The posterior anchors are distributed along a posterior line of the stomach wall beginning near the cardia region and toward the stomach exit. The anchor line and the stomach wall are drawn towards the posterior line of the stomach wall to reduce gastric volume.

### CROSS REFERENCE TO RELATED APPLICATIONS

This patent application claims priority to U.S. Provisional Patent Application Ser. No. 06/589,481 with an assigned filing date of Jul. 20, 2004.

### BACKGROUND OF THE INVENTION

1. Field of the Invention

This invention pertains to a method and apparatus for treating obesity. More particularly, this invention pertains to an apparatus and method for treating obesity with a less invasive procedure for reducing gastric volume.

2. Description of Prior Art

A. Obesity as a World-Wide Health Dilemma

Morbid obesity and its concurrent health risks (including diabetes, heart disease and other ailments) are of near-epidemic proportions in industrialized societies. A wide variety of treatments have been proposed and attempted to treat morbid obesity with a wide variety of efficacy and associated morbidity. These treatments include techniques to reduce stomach volume, alter gastric and intestinal motility, and alter the absorption of nutrients in the small intestine.

Clearly, obesity is a complex disease having physiologic, social and psychological components which are not fully understood. The complex nature and the enormous societal implication of obesity require a wide variety of treatment options be available to permit a physician to select a most appropriate option for a particular patient.

Even if all treatments were proven effective, no one treatment can meet the clinical needs presented by a diverse population. For example, current bariatric surgeries, such as the Roux-en-Y procedure as will be described, is not considered suitable for only so-called mildly obese patients (e.g., those with a Body Mass Index less than 35). Also, for extremely obese patients, operative risks may make this procedure undesirable.

Less invasive procedures (such as gastric banding, as will be described) have reduced surgical risk. Unfortunately, they suffer from reduced efficacy (and they are not without risks). Further, efficacy may be culturally biased. Namely, gastric banding studies show reduced efficacy in North American patients compared to European patients.

B. Selected Obesity Treatments
a. Surgical Options (Non-Device)
   i. Gastric Volume Reduction

Surgical approaches to gastric volume reduction include minimally invasive surgery, open surgery and endoscopic approaches to gastric volume reduction. Many such procedures have been tried and some have been abandoned due to lack of efficacy or unacceptable morbidity and mortality.

The gastric volume reduction procedures include vertical and horizontal gastroplasty in which sutures, staples or other fixation devices are used to join opposing surfaces of the stomach to create a reduced volume pouch and thereby reduce caloric intake.

Gastric stapling, as used herein, refers to staples or stitches in the stomach to reduce stomach size. These include horizontal gastroplasty and vertical gastroplasty.

Surgical gastric volume reduction is focused on reducing the fundus to induce satiety. Recent studies suggest antral volume more directly influences satiety. Strum, et al., “Energy Intake and Appetite are Related to Antral Area in Healthy Young and Older Subjects”, American Journal of Clinical Nutrition, 80(3), pp. 656-657 (2004).

Less invasive techniques for restricting the volume of the stomach also include a gastric partition in which the stomach wall is endoscopically cinched together to form a reduced size pouch. The cinching is performed using commercially available products such as the Bard EndoCinch™ and the Wilson-Cook Sew-Right™ cinching equipment. Such surgical equipment is generally described in U.S. Pat. No. 5,088,979 to Filipi et al. issued Feb. 18, 1992; U.S. Pat. No. 6,302,917 to Dua et al. issued Oct. 16, 2001 or PCT International Publication No. WO 01/89393 published Nov. 29, 2001.
ii. Surgeries with Malabsorptive Components

Treatments have been suggested and developed for reducing the amount of nutrient absorption in the small intestine (particularly in the upper and middle portions of the small intestine—the duodenum and jejunum, respectively).

In the duodenum, ducts from the pancreas and gall bladder discharge into the small intestine through small protrusions referred to as papilla. Commonly, pancreatic exocrine secretions (“PES”) flow from the pancreas in a pancreatic duct. Similarly, bile from the gall bladder flows through a bile duct. These ducts merge to form a common duct with discharges through a papilla into the duodenum. In some patients, the bile duct and pancreatic duct do not merge. They separately discharge into the duodenum at separate papilla which, usually, is in close proximity to one another.

Techniques to reduce nutrient absorption (commonly referred to as malabsorption treatments) include drug therapies for reducing lipids absorption. Such drug therapies have uncomfortable side effects, which can discourage a patient from complying with the drug therapy.

Other malabsorption treatments include surgical techniques for rerouting the intestinal system to bypass an extended portion of the small intestine. These include a so-called jejunoileal bypass. Not commonly used due to unacceptable mortality rates, a jejunoileal bypass would result in effective weight loss. Other techniques include the gastric bypass (or Roux-en Y) and duodenal switch. In both of these procedures, a large segment (e.g., in excess of 100 cm) of the small intestine (including the duodenum) are bypassed so that food content is rerouted from a small pouch formed in the upper portion of the stomach to the jejunum. As a result, the absorptive length of the small intestine is significantly shortened thereby reducing the amount of nutrients which are absorbed into the body and which support or lead to weight gain. Unfortunately, the foregoing surgical procedures are extremely invasive and, for the most part, not reversible.
b. Implantable Mechanical Devices
   i. Gastric Volume and Delayed Gastric Emptying
      1. Gastric Banding

Less invasive techniques are suggested for placing a band (referred to as LAP bands) around an upper portion of the stomach to act as a belt to reduce the size of the stomach and create a small passageway (a stoma) from a small upper pouch to the remainder of the stomach. An example of a LAP band is shown in U.S. Pat. No. 5,266,429 to Kuzmak dated Jul. 13, 1993. LAP bands and other gastric bandings are disclosed in Schauer, et al, “Surgical Management of Gastroesophageal Reflux Disease in Obese Patients”, Seminars in Laparoscopic Surgery, Volume 8, Number 4, pages 256-264 (2001). Such LAP bands wrap around a portion of the fundus to create a greatly reduced volume portion of a fundus above the LAP band. Such bands create an upper chamber above the band to create a sensation of satiation after consuming only a small volume of food. See also, U.S. Pat. No. 5,549,621 to Bessler et al., dated Aug. 27, 1996; U.S. Pat. No. 5,226,429 to Kuzmak dated Jul. 13, 1993 and U.S. Pat. No. 4,592,339 to Kuzmak et al. dated Jun. 3 1986.
2. Intra-Gastric Balloons

Other techniques for reducing gastric volume size include placement of obstructions within the stomach. These include intra-gastric balloons which are filled with saline to reduce the effective volume of the stomach. Examples of such balloons or other intragastric devices include those shown in U.S. patent application publication No. US 2001/0037127 to de Hoyos Garza published Nov. 1, 2001 (describing a percutaneous intragastric balloon to treat obesity); U.S. patent application publication No. 2002/0055757 to Torre, et al., published May 9, 2002; U.S. patent application publication No. 2004/0093091 to Gannoe, et al., published May 13, 2004 (describing an anchored intragastric balloon); U.S. patent application publication No. 2004/004357 to Gannoe, et al., published Mar. 4, 2004 (describing various techniques for retaining an intragastric balloon in a location in the stomach) and U.S. patent application publication No. 2003/0158601 Silverman published Aug. 21, 2003.
3. Pyloric Narrowing

U.S. patent application publication No. 2004/0019388 to Starkebaum published Jan. 29, 2004 describes treating obesity by injecting bulking agents into the pylorus. U.S. patent application publication No. 2004/0037865 to Miller published Feb. 26, 2004 describes various techniques to narrow the pylorus to slow gastric emptying to treat obesity. For example, the '865 application describes injecting bulking or stiffening agents into the pylorus. The application also describes ablation or scarring to narrow the pylorus as well as suturing the pylorus to narrow it.

U.S. patent application publication No. 2004/0089313 to Utley, et al., May 13, 2004 describes treating the pylorus to slow or meter gastric emptying. The '313 application describes treating tissue at the pylorus with an agent to tighten tissue or with a bulking agent. The application also describes treating the pylorus with an agent to interrupt afferent nerve impulses that trigger transient sphincter relaxation. The application also describes applying ablative energy to the pylorus, using magnets to tighten the pylorus or placing bands around the pylorus.

U.S. patent application publication No. US 2002/0188354 to Peghini published Dec. 12, 2002 teaches a device to treat obesity by obstructing the gastric outlet at the pylorus. The '354 application describes a device for obstructing the pylorus to create a sensation of satiety. The obstruction is a sandglass shaped device having bulges placed on opposite sides of the pylorus (one in the stomach, the other in the small bowel) with a narrow bridge spanning the pylorus. The device is formed of plastic and endoscopically delivered and fluid filled.

U.S. patent applications Publication Nos. US 2005/0033331 and US 2005/0055039 describe pylorus obstruction devices and methods.
4. Other

There fore-going description of prior art patents is not intended to be exhaustive. In the patent literature, there are many other suggestions for treating obesity. For example, U.S. patent application Publication No. 2003/0158601 to Silverman, et al., published Aug. 21, 2003 describes injections of implants in the stomach wall near the pylorus to inhibit gastric emptying. U.S. patent application Publication No. 2004/0172142 to Stack, et al., published Sep. 2, 2004 describes covered stent-like structures in the antrum and duodenum and bridging the pylorus.
ii. Devices to Promote Malabsorption

Less invasive techniques for restricting absorption have been suggested. They include bariatric sleeve devices such as those disclosed in US Patent Application Publication Nos. 2004/0092892 to Kagan, et al., published May 13, 2004 and 2004/0107004 to Levine, et al., published Jun. 3, 2004. In these techniques, sleeves are passed through the duodenum so that chyme (the contents of the intestines) are passed through the sleeve and do not interact with the absorptive walls of the intestine. The sleeves may be perforated to permit some of the chyme material to pass through the walls of the small intestine and be absorbed as nutrients. The sleeve of the '004 application includes a stent in the pylorus. The stent keeps the pylorus permanently open to induce a so-called “dumping syndrome”.

The bypass of the duodenum results in reduced absorption of desired nutrients (e.g., calcium) as well as undesirable nutrients (such as fat). Particularly, the loss of calcium absorption is significant since such loss can lead to osteoporosis.

A suggestion has been made to divert the digestive enzymes from the pancreas past the duodenum. Such a suggestion is shown in the afore-mentioned US Patent Application Publication No. 2004/0092892. In an embodiment of the '892 application, a tube is placed through the papilla and into the ducts of the gall bladder and the pancreas. A distal end of the tube is positioned significantly distal to the papilla such that pancreatic exocrine secretion and bile are diverted significantly distally to the papilla resulting in a reduction of absorption.

While pancreatic diversion is scientifically interesting, cannulation of the pancreatic duct carries significant risks. Such cannulation of the pancreatic duct has been performed in endoscopic retrograde cholangiopancreatography (ERCP). Patients under-going ERCP and/or related procedures are known to have a higher likelihood of developing pancreatitis. It has been reported that the incidence of post-ERCP pancreatitis can be as high as 28%. Fazel et al., “Prophylactic Pancreatic Duct Stenting: A Panacea”, Gastroenterology, Vol. 124, No. 4, pp. 1274-1275 (2003). Pancreatitis is a very serious disease which can be fatal.
c. Electrical Neural Stimulation

There have been a number of suggestions to treat obesity by applying electrical stimulation. For example, two patents assigned to Cyberonics, Inc. describe purported obesity treatments involving stimulation signals applied to the vagus nerve to up-regulate vagal activity to near a so-called “retching threshold”. These are U.S. Pat. Nos. 6,587,719 and 6,609,025.).

U.S. Pat. No. 6,615,084 to Cigaina dated Sep. 2, 2003 (assigned to Transneuronix) delivers direct smooth muscle stimulation to the stomach through a laparoscopically placed lead connected to an implantable pulse generator. Similarly, U.S. Pat. No. 5,423,872 to Cigaina dated Jun. 13, 1995 describes placing electrodes on the abdominal wall.

A number of patents and patent applications are assigned to Intrapace Inc pertaining to an endoscopically delivered direct stimulation device for the treatment of obesity. Examples of these are U.S. Pat. No. 6,535,764; US 2003/0167025; US 2003/016024; WO 02/087657; and WO 02/089655.

Also, proposed stimulation therapies include technologies to provide direct gastric stimulation to create a ‘banding’ effect on the stomach formed by contracted muscle. U.S. Pat. No. 6,571,127 to Ben-Haim et al., dated May 27, 2003 describes applying a field to a GI tract to increase the force of contraction. U.S. Pat. No. 6,600,953 to Flesher et al., dated Jul. 29, 2003 describes a set of electrodes on the stomach which cause a contraction to decrease a cross-section of the stomach.
d. Electrical Neural Block

Recent novel treatments include vagal modulation to block neural impulses on the vagus nerve to down-regulate pancreatic exocrine secretion production as well as alter gastric accommodation. Such treatments are shown in U.S. Patent Application Publication No. 2004/0172086 A1 to Knudson, et al.

### SUMMARY OF THE INVENTION

According to a preferred embodiment to the present invention, a method and apparatus are disclosed for reducing gastric volume. The method includes deploying an endoscope into the stomach through the esophagus of a patient. A plurality of anterior anchors are affixed to an anterior wall of the stomach. The anterior anchors are distributed along anterior lines of the stomach while being proximate the cardia region and extending toward the stomach exit. The method further includes a fixing of plurality of posterior anchors to the posterior wall of the stomach. The posterior anchors are distributed along a posterior line of the stomach wall beginning proximate the cardia region and extending toward the stomach exit. The anterior line of the stomach wall is drawn toward the posterior line of the stomach wall to reduce gastric volume. The apparatus of the present invention includes a stem with an anchor end and a connector end. One or more anchor members are provided proximate the anchor end. The anchor members have an insertion configuration and a deploy configuration. In the insertion configuration, the anchors are aligned with the stem. In the deploy configuration the anchors are out of alignment with the stem. A tip according to the present invention includes a plurality of stomach wall anchor devices.

### BRIEF DESCRIPTION OF THE DRAWINGS

FIG. 1 is a view of the gastro-esophageal region of a human subject.

FIG. 2A is a side elevation view of one exemplary device that may be used in connection with the present invention in an insertion configuration.

FIG. 2B is the view of FIG. 2A showing the device in a deployed configuration.

FIG. 3 is a partial cross-sectional view of two pairs of anchors from a gastric reduction kit deployed in opposing anterior and posterior walls of a stomach.

### DETAILED DESCRIPTION OF THE PREFERRED EMBODIMENT

The present invention provides endoscopic gastric reduction and/or reshaping devices and methods for reducing/reshaping the stomach size to suppress appetite. The devices and methods involve drawing the opposing anterior and posterior walls of the stomach together along all or part of the length of the stomach, beginning proximate the cardia region and extending towards the stomach exit (e.g., the pyloris and/or the pyloric sphincter).

It may be preferred to reduce the size of the stomach volume to a volume considered to be the fasting volume, e.g., 200 cubic centimeters (cc) to achieve desirable appetite suppression.

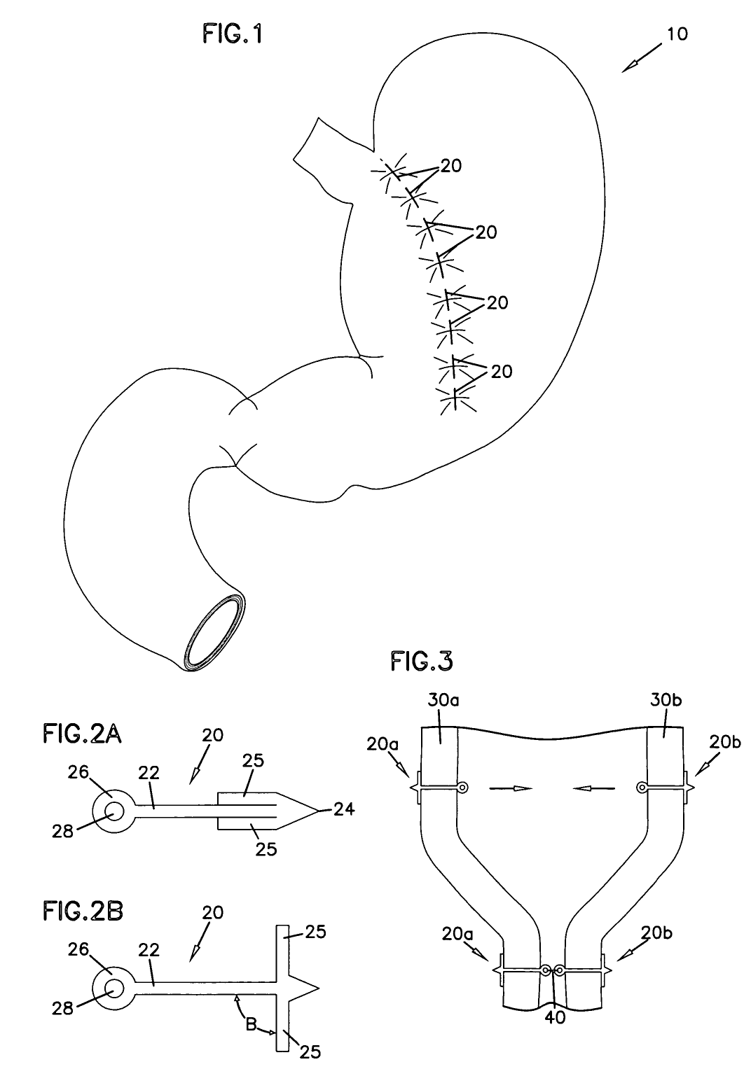

FIG. 1 is an anterior view of a stomach 10 on which an anterior line of stomach wall anchors 20 is located on the exterior surface of the stomach. The anchors 20 define the anterior line, which may preferably begin proximate the cardia region (near the entrance of the esophagus to the stomach 10) and extend towards the stomach exit near the proximal end of the small intestine.

Although only the anterior surface of the stomach 10 is depicted in FIG. 1, it will be understood that a complementary set of stomach wall anchors is provided along the 5 posterior surface of the stomach 10 for connection to the anterior set of anchors 20 seen in FIG. 1. FIG. 3 shows anchors 20a, 20b in opposing surfaces of the anterior wall 30a and the posterior wall 30b.

Each of the anterior anchors 20 is preferably urged or drawn towards a corresponding anchor in the posterior stomach wall, such that the gastric volume available for food entering the stomach 10 is reduced. The depicted anterior line extends only partially along the length of the stomach 10, although it will be understood 10 that the line may alternatively extend along the entire length of the stomach down towards the stomach exit.

In some instances, it may be preferred that the line of anchors isolates the fundus (the upper portion of the stomach) and a significant portion of the body of the stomach from the available gastric volume. In other instances, such isolation may not be complete, i.e., it may still be possible for food to enter the fundus and/or remainder of the body of the stomach after moving past the line of anchors.

Because of the reduced available gastric volume, the satiety sensors located within the cardia region are triggered when the subject eats a smaller amount of food than if the subject's entire gastric volume is available. Such early triggering preferably results in a reduction in the subject's appetite, leading to a reduced volume of food consumption. That reduced food consumption preferably leads to weight loss as the subject presumably consumes fewer calories.

The anchors 20 may take a variety of configurations. Functionally, the anchors 20 are preferably capable of being deployed into the subject's stomach through a device such as endoscope inserted through the subject's esophagus. One example of a potentially suitable anchor 20 is schematically depicted in FIG. 2A where the anchor 20 is depicted in an insertion configuration. FIG. 2B shows the anchor 20 in a deployed configuration.

The anchor 20 includes a stem 22 having an insertion tip 24 and anchor members 25 proximate the anchor end of the anchor device 20. The anchor members are elongated members having one end hinged or otherwise pivotally connected to the stem 22 near the tip 24. The anchor device 20 also includes a connector end 26 including, in the depicted embodiment, an eyelet 28 formed therein.

In the insertion configuration (FIG. 2A), it is preferred that the anchor members 25 be substantially aligned with the longitudinal axes of the anchor members parallel to the stem 20 and with the anchor members 25 lying in side-by-side abutting relation to the stem 20. In this alignment, the profile of the anchor 20 is sufficiently small to assist in insertion of the anchor 20 through the stomach wall.

In the deployed configuration (FIG. 2B), the anchor members 25 are preferably not aligned with the stem 22. Instead, the anchor members 25 are pivoted outwardly and way from the stem 22 to define an angle B between the stem 22 and the anchor members 25. This configuration prevents or reduces the likelihood of the anchor 20 pulling back into the stomach through the opening created for insertion of the anchor 20. Such openings may be self-formed by the tip 24 being advanced through the stomach wall or may be pre-formed by any suitable tool. In the depicted embodiment, the anchor members 25 form right angles with the stem (angle B), although smaller acute angles between the anchor members 25 and the stem 22 may also provide the desired functionality of anchoring the anchor 20.

The anchors 20 may be moved from the insertion configuration to the deployed configuration by any suitable techniques known to those of skill in the art. For example, the anchor 20 (or portions thereof) may be constructed of shape-memory materials such as, e.g., NITINOL, etc. The anchor members 25 may be hinged to the stem 22 or may be elastically deformed to the position of FIG. 2A and released to return to a rest state of FIG. 2A in response to elastic bias.

Also, the anchors 20 may include as few as one anchor arm, or two or more anchor arms as desired. Further, different configurations may be provided, for example, the anchor ends of the anchors may employ other structures that result in an increase in the cross-sectional area of the anchor end to reduce pull-out of the anchors. In other configurations, the anchors may include barbs or other structures along the stem 22 to reduce pull-out of the anchors.

FIG. 3 depicts one embodiment of a gastric reduction kit in use. The kit includes anchors 20a in the anterior wall 30a of the stomach and anchors 20b deployed in the posterior wall 30b of the stomach. After deployment, the anchors 20a and 20b are drawn towards each other, thus drawing the anterior wall 30a and the posterior wall 30b together. The “contraction” arrows near the uppermost pair of anchors 20a and 20b depict the direction of the contraction forces.

The lowermost pair of anchors 20a and 20b have been drawn together and are retained in that position using, in the depicted embodiment, a loop of suture material 40 extending through eyelets 28 provided in the connector ends of each of the anchors. It will be understood that the connector ends of each of the anchors 20a and 20b may take any suitable form other than that shown in FIG. 3 and that the connector ends of the anchors may be retained together by any suitable material, materials, or techniques, e.g., sutures, mechanical connectors (staples, threaded fasteners, wire loops, etc.). Furthermore, the contraction forces may be supplied by any suitable technique or techniques, e.g., a rotating take-up barrel with arms of lengths of suture material connected to opposing anterior and posterior anchors, such that rotation of the take-up barrel draws the anterior and posterior connectors towards each other. Other contraction forces may be supplied by lengths of suture material, rack-and-pinion devices, etc.

Adhesive Attachment Techniques and Systems

In addition to the methods in which attachment of the stomach walls is accomplished by primarily mechanical techniques and devices, it may be preferred to use adhesive materials in addition to mechanical fasteners (e.g., sutures, clips, staples, anchors, etc.). One potential point of failure when reshaping the stomach is maintaining the sutures/anchors within the gastric tissue. In addition, the use of adhesives could encourage tissue adhesion in reshaping the stomach and may result in a stronger attachment. Also, the combination of adhesive and mechanical fastening may result in a more uniform, tighter seal across the tissue being opposed. Mechanical attachment techniques may produce seals that are periodic with gaps between discrete attachment points. These gaps result in an attachment line that may be 20 weakened and potentially subject to failure.

In some instances, it may be possible to provide for gastric reduction/reshaping with the use of adhesive compositions alone, i.e., in the absence of mechanical connectors left in place after delivery of the adhesive compositions.

The present invention may involve treating two or more sites within the stomach with a tool designed to damage and/or remove the mucosal lining of the stomach (if required), apply an adhesive composition, and secure the treated areas together. The treated areas may preferably be generally opposed to each other across the stomach. After application of the adhesive composition, it may be preferable to bring the areas into physical association for a defined period of time. For example, the areas to be attached may be held together with a series of sutures, clips, staples, anchors, or other devices.

The treated areas may preferably eventually adhere to each other through proliferation of the tissues underlying the mucosal layer. This tissue adhesion would 5 preferably effectively shrink the stomach to a smaller volume and preferably cause the patient to experience satiety much earlier in the course of eating.

It may be preferred to prepare the stomach wall for application of the adhesive composition. Depending on the properties of the adhesive composition, it may be preferred to damage the mucosal layer before or at the same time as the adhesive composition is applied. Tissue may be prepared by, e.g., cutting the tissue with a blade or other device, damaging the tissue with a heating element, optical energy (e.g., laser, etc.), ultrasonic energy, etc. In the case of heat damage, the thermal energy may preferably be provided in specific locations and at frequencies that may define the depth of damage, creating areas of tissue damage, and resultant scarring, that if deep enough in the tissue could potentially prevent the stomach from expanding. Tissue site preparation may also be accompanied by use of, e.g., a sclerotic agent.

Adhesive compositions used in connection with the present invention may have a variety of properties and characteristics. One embodiment could be a thermoset adhesive, preferably allowing good flow and fluidity while providing the opportunity for controlled curing through the application of thermal energy. Other adhesive compositions may be used in connection with the present invention, e.g., light curable adhesives, moisture-curable adhesives, etc.

The adhesive compositions used in connection with the present invention may optionally be combined with a growth promoting agent (angiogenic factor, growth factor, fibrosis promoting factor, etc.) and/or a sclerotic agent. The adhesive composition may be permanent or biodegradable. In some embodiments, the adhesive composition need only function for an effective amount of time, e.g., until tissue adhesion (typically about two weeks).

The sutures, clips, staples, anchors, or other devices used in addition to adhesive compositions may themselves be permanent or biodegradable. In some instances, it may be preferred to coat the devices with the adhesive composition in place of or in addition to supplying the adhesive composition alone. As mentioned above, the attachment devices may need to function for a limited period of time, e.g., until tissue adhesion occurs (typically about two weeks).

The attached sites may prefera0bly be any opposed or adjacent areas of the stomach that will accomplish the desired reshaping of the stomach. One preferred method would treat a line extending generally from the fundus to the antrum, e.g., parallel to the line of the greater curve of the stomach. An example of one line of attachment is depicted in FIG. 1. Although it may be preferred that the line extend for substantially the entire length of the stomach, the methods of the invention may involve forming one or more shorter lines of attachment. It may be preferred that the reshaping leave a “tube” running from the esophagus to the antrum (that may preferably have a volume of about 200 cubic centimeters)

It may be preferred that any devices used in connection with the present invention be endoscopically deployed, although surgical approaches may alternatively be used. In addition to site preparation devices, e.g., blades, rasps, heating elements, optical fibers/emitters, ultrasonic transducers/waveguides, etc., the present invention may also involve the use of injection devices (e.g., needles) or other devices to deliver the adhesive composition by, e.g., spraying, brushing, dropping, extruding, etc. The adhesive composition may be delivered behind or adjacent to the mucosal layer at the attachment site during the site preparation. The adhesive composition may be delivered before, during, and/or after site preparation and mechanical attachment of the tissue. Site preparation, mechanical attachment, and adhesive delivery may preferably be accomplished by a single, integrated device. Alternatively, two or more of the functions of site preparation, attachment, and adhesive delivery may be performed by different devices, e.g., one device may be used for site preparation, another device for adhesive composition delivery, and another device may be used for tissue attachment.

The documents identified below may describe devices, methods and/or adhesive compositions that may potentially be useful in connection with the present invention.

Various attachment devices may be known to those of skill in the art. Some potentially suitable devices, systems and methods that may be used in connection with the present invention may be described in, e.g., U.S. Patent Publication Nos. US 2003/0181924 (Yamamoto et al.) and US 2004/0034369 (Sauer et al.); U.S. Pat. No. 6,736,828 B1 (Adams et al.); and International Publication No. WO 01/66108 (Gambale et al.).

U.S. Patent Publication No. 2003/0220660 A1 (Kortenbach et al.) describes a system and methods of using tissue fastening devices in combination with sclerosing agents to promote tissue adhesion.

U.S. Patent Publication No. 2003/0191476 A1 (Smit) discloses devices and methods of coating portions of the small intestine with a tissue sealant.

International Publication Nos. WO 03/105661 A2 (Huang) and WO 93/21905 (Shaked et al.) and U.S. Pat. Nos. 5,173,301 (Itoh et al.) and 4,806,614 (Matsuda et al.) describe adhesive compositions that may potentially be useful in connection with the present invention.

As used herein and in the appended claims, the singular forms “a,” “and,” and “the” include plural referents unless the context clearly dictates otherwise. Thus, for example, reference to “an anchor member” includes a plurality of anchor members and reference to “the anchor” includes reference to one or more anchors and equivalents thereof known to those skilled in the art.

All references and publications cited herein are expressly incorporated herein by reference in their entirety into this disclosure. Illustrative embodiments of this invention are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

The above specification, examples and data provide a complete description of the manufacture and use of the composition of the invention. Since many embodiments of the invention can be made without departing from the spirit and scope of the invention, the invention resides in the claims hereinafter appended.

We claim:

1. A method for reducing gastric volume, the method comprising:
deploying an endoscope into a stomach through the esophagus;
affixing a plurality of anterior anchors to the anterior wall of the stomach, wherein the anterior anchors are distributed along an anterior line of the stomach wall beginning proximate the cardia region and extending toward the stomach exit;
affixing a plurality of posterior anchors to the posterior wall of the stomach, wherein the posterior anchors are distributed along a posterior line of the stomach wall beginning proximate the cardia region and extending toward the stomach exit;
drawing the anterior line of the stomach wall towards the posterior line of the stomach wall, whereby gastric volume is reduced.

2. A stomach wall anchoring device comprising:
a stem comprising an anchor end and opposing connector end;
one or more anchor members proximate the anchor end, wherein the one or more anchor members each comprise an insertion configuration and a deployed configuration, wherein in the insertion configuration the one or more anchor members are aligned with the stem, and further wherein in the deployed configuration the one or more anchor members are not aligned with the stem; and a connector proximate the connector end.

3. A device according to claim 2, wherein the connector comprises an eyelet.

4. A device according to claim 2, wherein the connector comprises a hook.

5. A gastric reduction kit comprising:
a plurality of stomach wall anchor devices, wherein each of the anchor devices comprises an anchor end and a connector end;
one or more contraction devices adapted to engage the connector ends of the anchor devices, whereby the anterior and posterior stomach walls can be urged towards each other.

6. A kit according to claim 5, wherein the anchor devices comprises a stomach wall anchoring device comprising:
a stem comprising an anchor end and opposing connector end;
one or more anchor members proximate the anchor end, wherein the one or more anchor members each comprise an insertion configuration and a deployed configuration, wherein in the insertion configuration the one or more anchor members are aligned with the stem, and further wherein in the deployed configuration the one or more anchor members are not aligned with the stem; and a connector proximate the connector end.

7. A kit according to any of claim 5, wherein the one or more contraction devices comprise suture material.

8. A kit according to any of claim 5, wherein the one or more contraction devices comprises a rotating barrel take-up.

9. A method according to claim 1, further comprising providing an adhesive composition on the stomach wall.

10. A method according to claim 9, further comprising preparing the stomach wall before providing the adhesive composition.

11. A kit according to claim any of claim 5, further comprising an amount of a biocompatible adhesive composition.
